(19) United States
(12) Patent Application Publication
(10) Pub. No.: US 2022/0111162 A1
(43) Pub. Date: Apr. 14, 2022

(54) APPARATUS, SYSTEM, AND METHOD FOR FACILITATING INTRANASAL TREATMENT OF A PATIENT

(71) Applicant: Lido Ventures LLC, Woods Cross, UT (US)

(72) Inventors: Stephen J. Eldredge, South Jordan, UT (US); Dean L. Howes, North Salt Lake, UT (US); Wade Cooper, Ann Arbor, MI (US)

(21) Appl. No.: 17/389,152

(22) Filed: Jul. 29, 2021

Related U.S. Application Data
(60) Provisional application No. 63/058,075, filed on Jul. 29, 2020.

(51) Int. Cl. A61M 15/08; A61K 31/167; A61K 9/00; A61K 31/522; A61K 47/26

(52) U.S. Cl. CPC A61M 15/08; A61K 31/167; A61M 2202/048; A61K 31/522; A61K 47/26; A61K 9/0043

(57) ABSTRACT

An apparatus is provided for facilitating intranasal treatment of a patient's sphenopalatine/pterygopalatine recess. The apparatus includes an intranasal spray delivery device configured to contain a spray composition, atomize the spray composition, and deliver the atomized spray composition to the sphenopalatine/pterygopalatine recess of the patient. In certain examples, the spray composition includes about 2.5% lidocaine and about 0.10% maltoside. A method is also provided and includes providing, in a solution, about 2.5% lidocaine, providing, in the solution, about 0.10% maltoside, and placing the solution in an intranasal spray delivery device configured to contain the solution and deliver the solution to the sphenopalatine/pterygopalatine recess of the patient.

### CROSS-REFERENCES TO RELATED APPLICATIONS

This application claims the benefit of U.S. Provisional Patent Application No. 63/058,075 entitled “APPARATUS, SYSTEM, AND METHOD FOR FACILITATING INTRANASAL TREATMENT OF A PATIENT” filed on Jul. 29, 2020 for Stephen J. Eldredge, et al., which is incorporated herein by reference.

### FIELD

This application relates generally to intranasal treatment of a patient with medication, and in particular, relates to intranasal spray delivery of a medication.

### BACKGROUND

Nasal delivery of medication/anesthesia is often performed in many medical offices during many types of examinations and treatments. Doctors may use many different devices and methods for the delivery of the medication, which includes transdermal or transmucosal pastes, creams, liquids, solids, and semisolids. The methods that doctors use frequently rely on topical administration of medication using, for example, cotton swabs. Although there are intranasal sprays used by doctors, currently there is no over-the-counter intranasal spray device that allows for the self-administration of a medication such as lidocaine.

### BRIEF SUMMARY

An apparatus is disclosed for facilitating intranasal treatment of a patient's sphenopalatine/pterygopalatine recess. The apparatus includes an intranasal spray delivery device configured to contain a spray composition, atomize the spray composition, and deliver the atomized spray composition to the sphenopalatine/pterygopalatine recess of the patient. In certain examples, the spray composition includes about 2.5% lidocaine and about 0.10% maltoside.

In certain examples, the spray composition includes about 2% of a stimulant, which may be caffeine. In certain examples, the maltoside is selected from the group of n-Decyl-β-D-maltopyranoside, n-Dodecyl-3-D-maltopyranoside, and 6-Cyclohexyl-1-hexyl-β-D-maltopyranoside. In other certain examples, the maltoside is 1-O—N-dodecyl-beta-D-maltopyranoside.

The spray composition, in certain examples, also includes sodium chloride, a buffering agent, a viscosity agent, and a stability agent. In certain examples, the intranasal spray delivery device comprises a tri-turbinate nasal pump.

A method is also disclosed. In certain examples, the method includes providing, in a solution, about 2.5% lidocaine, providing, in the solution, about 0.10% maltoside, and placing the solution in an intranasal spray delivery device configured to contain the solution and deliver the solution to the sphenopalatine/pterygopalatine recess of the patient. The method may also include providing, in the solution, a stimulant.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIGS. 1 and 2 are illustrations of environments in which the present subject matter may be practiced;

FIG. 3 illustrates a cotton-tipped applicator of the prior art;

FIG. 4 is a side view diagram of one example of an intranasal spray device for the self-treatment of headaches, according to examples of the subject disclosure; and

FIG. 5 is a schematic flowchart diagram illustrating one example of a method of preparing an anesthetic solution for atomization, according to examples of the present disclosure.

### DETAILED DESCRIPTION

Reference throughout this specification to “one embodiment,” “an embodiment,” or similar language means that a particular feature, structure, or characteristic described in connection with the embodiment is included in at least one embodiment of the present subject matter. Thus, appearances of the phrases “in one embodiment,” “in an embodiment,” and similar language throughout this specification may, but do not necessarily, all refer to the same embodiment.

Furthermore, the described features, structures, or characteristics of the subject matter may be combined in any suitable manner in one or more embodiments. In the following description, numerous specific details are provided for a thorough understanding of embodiments of the subject matter. One skilled in the relevant art will recognize, however, that the subject matter may be practiced without one or more of the specific details, or with other methods, components, materials, and so forth. In other instances, well-known structures, materials, or operations are not shown or described in detail to avoid obscuring aspects of the subject matter.

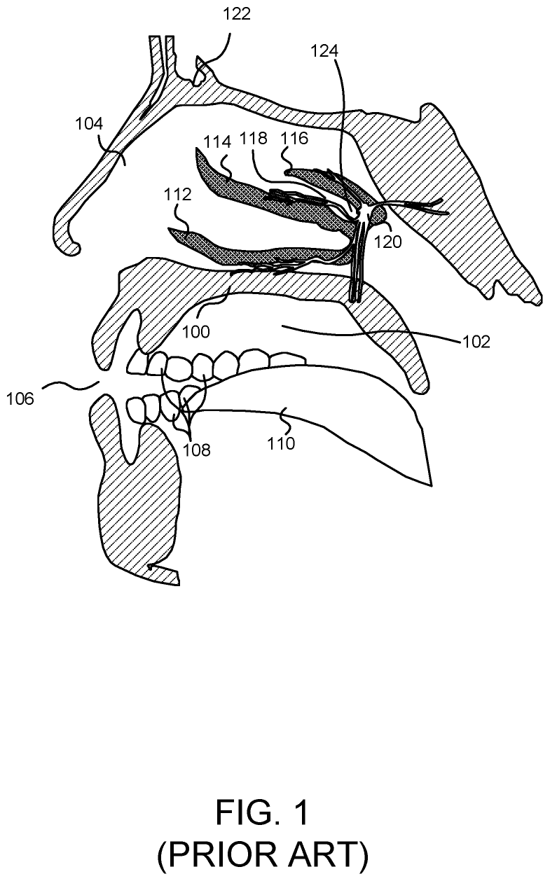

FIG. 1 is an illustration of one environment in which the present subject matter may be practiced. In particular, FIG. 1 depicts a cutaway view of the anatomical features of a typical human nasal cavity. One skilled in the art will recognize that certain anatomical features and structures of the human nasal cavity have been omitted to avoid obscuring the structures relevant to the practice of the current subject matter. To help orient the reader, the mouth 106 is illustrated with teeth 108 and tongue 110. The anatomical structures relevant to one practice of the current subject matter include the palate 100 which separates the oral cavity 102 from the nasal cavity 104, the inferior sinus turbinate 112, the middle sinus turbinate 114 and the superior sinus turbinate 116 as well as the nasal bone 122. The middle sinus turbinate 114 and superior sinus turbinate 116 define the sphenopalatine/pterygopalatine recess 118. Deep within the sphenopalatine/pterygopalatine recess 118 at the posterior 124 of the sphenopalatine/pterygopalatine recess 118 lies the sphenopalatine/pterygopalatine ganglia 120.

One skilled in the art will recognize that the medical community is not uniform in the terminology with regard to the sphenopalatine or pterygopalatine ganglia. Certain practitioners use sphenopalatine while others use pterygopalatine. Therefore, the present description will refer to the ganglia labeled 120 as the sphenopalatine/pterygopalatine ganglia 120. Similarly, the recess labeled 118 will be referred to as the sphenopalatine/pterygopalatine recess 118. However, this terminology is in no way limiting on the structure for which the present subject matter is intended. Where practitioners or scientist differentiate between the sphenopalatine ganglia or the pterygopalatine ganglia, the present disclosure will be understood to apply to either structure.

Sympathetic pain is a type of nerve pain that arises due to abnormalities in the function of the sympathetic nervous system. The majority of the “treatment resistant” headache population in the world suffers from what is now properly identified as “Sympathetic Mediated Cephalgia” a particular type of sympathetic pain. With sympathetic pain an abnormality in a group of nerves called a ganglion cause pain to an organ or body region. To treat sympathetic pain physicians can block a ganglion with the injection of medication into a specific area of the body. To therapeutically treat acute pain a physician injects a local anesthetic into the affected neuronal ganglion. This type of treatment may be referred to as a nerve block.

The sphenopalatine/pterygopalatine ganglia 120 is a neuronal structure located principally in the center of the head in the pterygopalatine fossa posterior to the middle turbinate 114. The sphenopalatine/pterygopalatine ganglia 120 comprises the largest cluster of sympathetic neurons in the head outside of the brain. The sphenopalatine/pterygopalatine ganglia 120 interfaces and directs nerve impulses to the majority of the head's autonomic or parasympathetic pathways. Therefore, any abnormality or injury to this structure may cause severe pain. A nerve block of the sphenopalatine/pterygopalatine ganglia 120 is effective in relief in a variety of pain conditions ranging from headache to lower back pain. Additionally, other disease processes such as headache disorders and other neurological conditions can be arrested, or improved by local anesthetic blockade, and/or other pharmacological augmentation or mechanical alteration of the sphenopalatine/pterygopalatine ganglia 120 and surrounding structures.

Unfortunately, because of the anatomical position of the sphenopalatine/pterygopalatine ganglia 120, the structure is very difficult to block with a local anesthetic solution. The anatomical location of the sphenopalatine/pterygopalatine ganglia 120 is dangerously close to many vital and delicate mid brain structures. Although direct needle placement can be employed under fluoroscopic guidance to administer anesthetic to the sphenopalatine/pterygopalatine ganglia 120, most practitioners will not attempt the procedure due to the technical difficulty and extreme dangers of an aberrant needle placement.

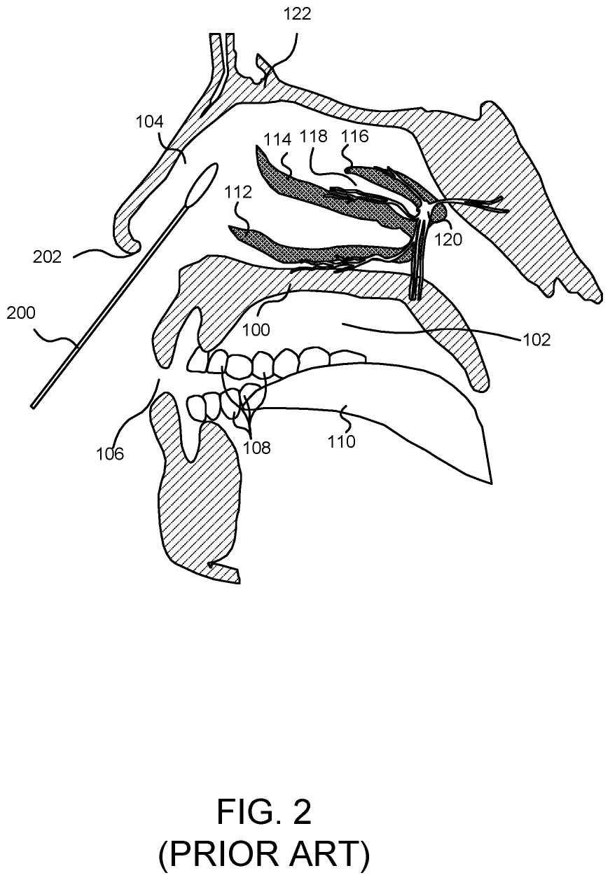

As shown in the prior art illustration depicted in FIG. 2, the sphenopalatine/pterygopalatine ganglia 120 lies deep within the sphenopalatine/pterygopalatine recess 118. Conventional methods undertaken by pain specialists, neurologists, and neurosurgeons, include the use of an eight-inch cotton-tipped applicator 200 saturated with a local anesthetic. Because a cotton-tipped applicator 200 is used, the procedure is referred to as the “Q-tip” procedure. The cotton-tipped applicator 200 is soaked in a vial of concentrated local anesthetic solution. In certain embodiments the anesthetic solution is lidocaine, cocaine, etidocaine or prilocaine, or other non-specified local anesthetic agents. The cotton-tipped applicator 200 is then advanced into the nostril 202 and through the nasal cavity 104. To reach the sphenopalatine/pterygopalatine ganglia 120 in the sphenopalatine/pterygopalatine recess 118, the cotton-tipped applicator 200 must be advanced into the nasal cavity 104 past the middle sinus turbinate 114 and into the sphenopalatine/pterygopalatine recess 118.

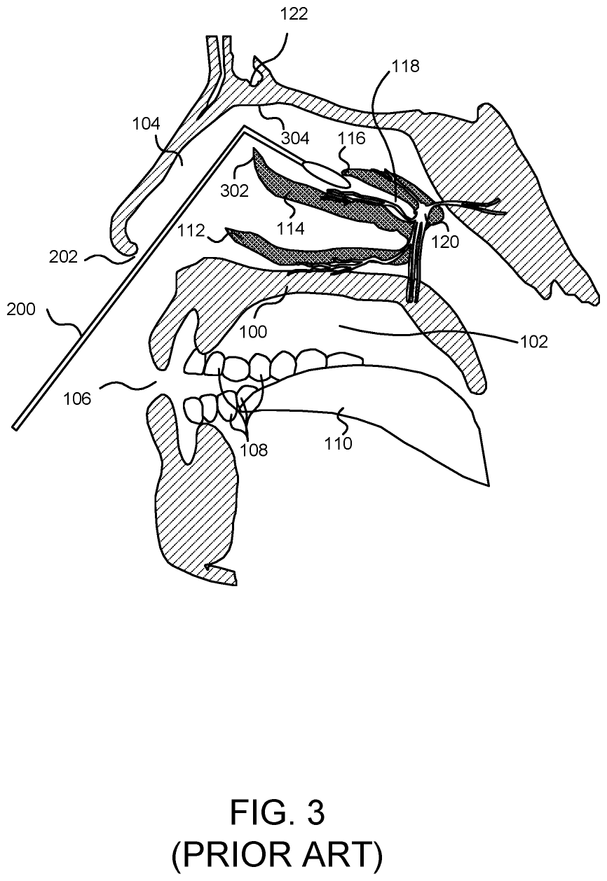

FIG. 3 illustrates the tortuous path the cotton-tipped applicator 200 of the prior art must traverse to reach the sphenopalatine/pterygopalatine recess 118. To perform the procedure the patient is placed in a supine position. The cotton-tipped applicator 200 is soaked in a vial of concentrated local anesthetic solution. The physician then inserts the cotton-tipped applicator 200 into the patient's nostril 202 and through the nasal cavity 104. Advancing the straight, rigid cotton-tipped applicator 200 into the sphenopalatine/pterygopalatine recess 118 can be difficult and painful for the patient as the cotton-tipped applicator 200 must be inserted almost parallel to the patient's face to clear the anterior ridge 302 of the middle sinus turbinate 114. The cotton-tipped applicator 200 must then make an almost 90° bend to avoid the inferior surface 304 of the nasal bone 122 and access the sphenopalatine/pterygopalatine recess 118. The cotton-tipped applicator 200 is left in the patient's sphenopalatine/pterygopalatine recess 118 for approximately 20 minutes to allow diffusion of the local anesthetic through the sinus mucosa to depolarize the sphenopalatine/pterygopalatine ganglia 120 to block nerve transmission.

The use of a straight and rigid cotton-tipped applicator 200 that must make some fairly tortuous directional changes around some very sensitive, richly vascular, friable, highly innervated structures complicates the procedure to the point that many practitioners will not attempt it. Known complications include extreme patient discomfort, nosebleeds and the complications associated with nosebleeds including venous-irritating nuisances, arterial hemorrhaging, aspiration, hematochezia or even death. Other complications include local anesthetic toxicity, seizure, iatrogenic foreign bodies such as a broken cotton-tipped applicator 200, sinus mucosal tears and infection.

Anesthetic blockade of any neuronal structure requires direct physical interaction between the anesthetic solution and the targeted tissue. Therefore, to work, the cotton-tipped applicator 200 must deliver the anesthetic solution directly to the sphenopalatine/pterygopalatine ganglion 120. The correct placement of the cotton-tipped applicator 200 is technically challenging and many practitioners simply miss the desired structure, the sphenopalatine/pterygopalatine ganglion 120 when attempting to perform the procedure. To help make the complicated bend required to reach the sphenopalatine/pterygopalatine recess 118 many practitioners will soak the top 2 inches of the cotton-tipped applicator 200 and manipulate the stem to render it flexible so that the patient is less agitated and bleeding risks are lessened. Even with a flexible cotton-tipped applicator 200 the procedure is difficult. Common failure placements include the inferior surface 304 of the nasal bone 122 and the anterior ridge 302 of the middle sinus turbinate 114. When the cotton-tipped applicator 200 is misplaced, a “wring-out” effect may occur wherein the anesthetic is wrung out of the cotton-tipped applicator before it is delivered to the sphenopalatine/pterygopalatine ganglion 120 resulting in an ineffective procedure. Further, as discussed above, the rich vascular and neuronal structure of the nasal cavity 104 makes any misplacement of the cotton-tipped applicator 200 both dangerous and painful.

For these reasons described above, self-administration of an anesthetic solution is not a possibility using intranasal devices that require physical application of the anesthetic. The administration of anesthetic in solution form requires a highly skilled medical professional to directly target and apply the anesthetic solution to the targeted tissue. Beneficially, the below described device and atomizable anesthetic solution allows self-administration of an anesthetic with the ability to achieve total mucosal delivery of the local anesthetic agent to the sphenopalatine/pterygopalatine recess 118.

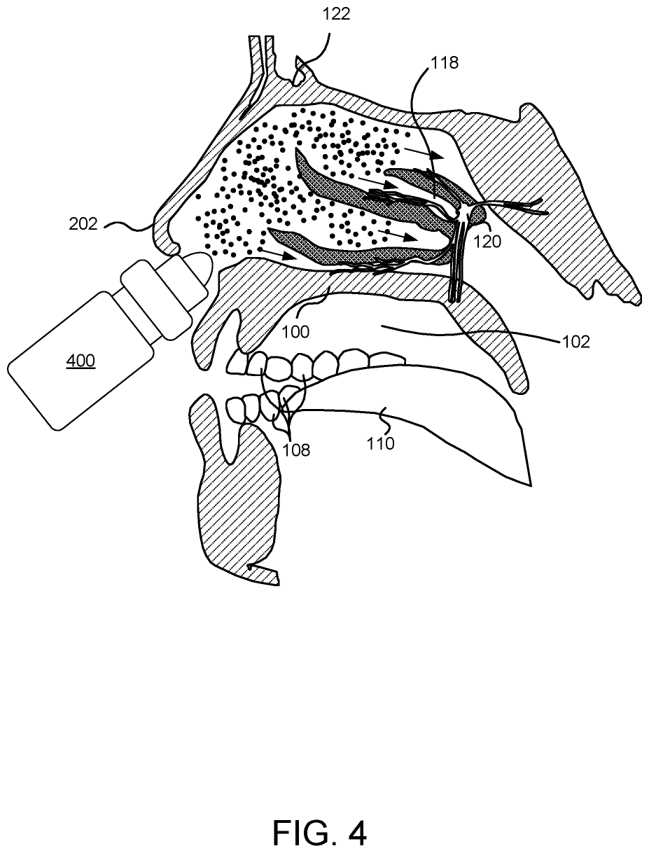

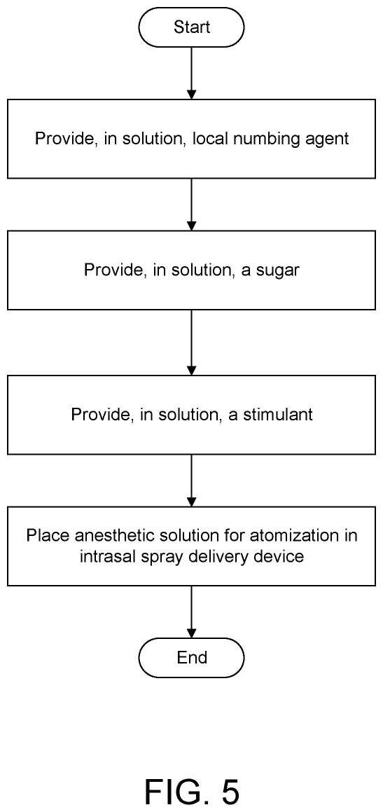

FIG. 4 is a side view diagram of one example of an intranasal spray device 400 for the self-treatment of headaches, according to examples of the subject disclosure. The intranasal spray device (hereinafter “device”) 400, in certain examples is a tri-turbinate nasal pump configured to atomize a medicament in solution form, and dispense the atomized anesthetic solution into the nasal cavity of a person suffering from a headache, including migraines. The device 400, filled with a medicament in solution form, beneficially allows for self-administration because the difficulty of targeted application of anesthetic in solution form is overcome by the atomized, spray form anesthetic solution. The composition of the anesthetic solution for atomization will be discussed in greater detail below with reference to FIG. 5. In certain examples, the device 400 is a pump action for atomizing and dispersing the solution. In alternative examples, the anesthetic solution may be in a pressurized aerosol formulation, and the device 400 is a pressurized aerosol device.

FIG. 5 is a schematic flowchart diagram illustrating one example of a method 500 of preparing an anesthetic solution for atomization, according to examples of the present disclosure. Although the method 500 is described as a series of steps, it is to be understood that the steps may be performed in a different order than described and depicted.

Generally, the anesthetic solution for atomization, according to examples of the subject disclosure, includes a local numbing agent, a sugar, and a stimulant in water. The water may be purified by distillation, deionization, or reverse osmosis filtering. The purified water beneficially minimizes variations of formulations which may occur if the water characteristics were not consistent. Additionally, purified water lessens the instances where microbial contamination occurs.

Preservatives may be added including, but not limited to, 0.1% benzalkonium chloride. As used herein, “%” concentration is based on a number of grams of the described constituent per 1000 mL of solution. “About” or “approximately” describes an amount in a range that covers ±10% of the described value. For example, “about 90%” refers to any value between and including 81% and 99%. Other preservatives may be used, including, but not limited to, saline, sodium hydrochloride, benzyl alcohol, methylparaben, propylparaben, etc. The preservative, in certain examples, is incorporated to maintain the solution free of pathogenic organisms.

The solution may also include a buffering agent to maintain a pH in a range that is suitable for application to the intranasal mucosal membranes. Generally, an acceptable pH value is in the range of about 4 to about 8. Buffering agents that can maintain acceptable pH values are well known, and include, but are not limited to, phosphate, citrate, and borate salt systems.

In certain examples, the local numbing agent (e.g., therapeutic or palliative agent) is intended to relieve headaches and migraines. Drugs useful for relieving pain resulting from headaches and migraines include, but are not limited to, lidocaine, benzocaine, tetracaine, dibucaine, cocaine, etidocaine, prilocaine, or other non-specified local anesthetic agents. In certain examples, the anesthetic solution to be atomized includes approximately 2% to 3% of lidocaine. In certain other examples, the anesthetic solution to be atomized includes approximately 2.5% of lidocaine (i.e., 2.25% to 2.75%). In certain other examples, the anesthetic solution to be atomized contains between 2.4% and 2.5% lidocaine.

In certain examples, the solution also includes a glycoside. The glycoside, for example, may be a maltoside with maltose as the functional group. Examples of glycosides include, but are not limited to, n-Decyl-β-D-maltopyranoside, n-Dodecyl-β-D-maltopyranoside, and 6-Cyclohexyl-1-hexyl-β-D-maltopyranoside. In certain examples, the solution (or spray composition) includes between about 0.05% and 0.20% 1-O—N-dodecyl-beta-D-maltopyranoside (“DDM”). In certain other examples, the solution includes about 0.10% DDM (i.e., between 0.09% and 0.11%). In yet other examples, the solution includes about 0.15% DDM.

Maltose acts as an excipient in the solution for the purpose of long-term stabilization, and facilitating drug absorption, and/or enhancing solubility. Other sugars useful as an excipient include, but are not limited to fructose, glucose, lactose, sucrose, maltodextrins, xylitol, etc. In certain examples, the solution contains between about 0.1% and 0.2% of the sugar. In certain other examples, the solution contains about 0.15% maltose.

In some examples, the stimulant or stimulating agent is caffeine. Alternatives to caffeine suitable for use in the present disclosure include, but are not limited to, . . . In certain examples, the solution contains about 2% caffeine.

In one example, a spray composition is prepared using the following ingredients (and other buffers and stabilizing agents), as described above, in amounts expressed in grams, together with 1000 mL of purified water:
2.5 g Lidocaine hydrochloride
0.15 g Maltose
2 g Caffeine
0.1 g Benzalkonium chloride

In certain examples, the spray composition may be prepared using the following ingredients (and other buffering, viscosity, and stabilizing agents), as described above, in amounts expressed in grams, together with 1000 mL of purified water:
2.4 g Lidocaine hydrochloride
0.0.10 g 1-O—N-dodecyl-beta-D-maltopyranoside (DDM)
0.01 g Benzylkonium Chloride
Sodium Chloride
Buffering agent
Viscosity agent
Stability agent

The method 500 of preparing the spray composition (i.e., anesthetic solution to be atomized) includes the steps of providing, at block 502, a local numbing agent in a solution. At block 504, providing a sugar. At block 506, a stimulant is provided. The local numbing agent, with or without the sugar and/or stimulant, are mixed together in solution. At block 508, the spray composition is placed in an intranasal spray delivery device (e.g., see FIG. 4). In certain examples, the spray composition is packaged in a 15 ml container with about 8 ml of spray composition. Each “spray” may deliver about 0.15 ml of spray composition. If each nostril may receive a dose, for example, every 5 minutes as needed. The 8 ml of spray provides for about 15 treatments per container. Beneficially, a person may self-administer this anesthetic spray composition without seeking out skilled medical personnel.

The present subject matter may be embodied in other specific forms without departing from its spirit or essential characteristics. The described embodiments are to be considered in all respects only as illustrative and not restrictive. The scope of the subject matter is, therefore, indicated by the appended claims rather than by the foregoing description. All changes which come within the meaning and range of equivalency of the claims are to be embraced within their scope.

What is claimed is:

1. An apparatus facilitating intranasal treatment of a patient's sphenopalatine/pterygopalatine recess, the apparatus comprising:
an intranasal spray delivery device configured to contain a spray composition, atomize the spray composition, and deliver the atomized spray composition to the sphenopalatine/pterygopalatine recess of the patient, where the spray composition comprises in solution:
about 2.5% lidocaine; and
about 0.10% maltoside.

2. The apparatus of claim 1, where the spray composition further comprises about 2% of a stimulant.

3. The apparatus of claim 2, where the stimulant is caffeine.

4. The apparatus of claim 1, where the maltoside is selected from a group consisting of n-Decyl-β-D-maltopyranoside, n-Dodecyl-β-D-maltopyranoside, and 6-Cyclohexyl-1-hexyl-β-D-maltopyranoside.

5. The apparatus of claim 4, where the maltoside is 1-O—N-dodecyl-beta-D-maltopyranoside.

6. The apparatus of claim 1, where the spray composition further comprises sodium chloride.

7. The apparatus of claim 6, where the spray composition further comprises a buffering agent.

8. The apparatus of claim 7, where the spray composition further comprises a viscosity agent.

9. The apparatus of claim 8, where the spray composition further comprises a stability agent.

10. The apparatus of claim 1, where the intranasal spray delivery device comprises a tri-turbinate nasal pump.

11. A nasal spray composition comprising:
water;
about 2.5% lidocaine; and
about 0.10% maltoside.

12. The nasal spray composition of claim 11, further comprising about 2% of a stimulant.

13. The nasal spray composition of claim 12, where the stimulant is caffeine.

14. The nasal spray composition of claim 11, where the maltoside is 1-O—N-dodecyl-beta-D-maltopyranoside.

15. The nasal spray composition of claim 11, further comprising sodium chloride.

16. The nasal spray composition of claim 15, further comprising a buffering agent.

17. The nasal spray composition of claim 16, further comprising a viscosity agent.

18. The nasal spray composition of claim 17, further comprising a stability agent.

19. A method of preparing an intranasal treatment for a patient's sphenopalatine/pterygopalatine recess, the method comprising:
providing, in a solution, about 2.5% lidocaine;
providing, in the solution, about 0.10% maltoside; and
placing the solution in an intranasal spray delivery device configured to contain the solution and deliver the solution to the sphenopalatine/pterygopalatine recess of the patient.

20. The method of claim 19, further comprising, providing, in the solution, a stimulant.
